(19) United States
(12) Patent Application Publication
(10) Pub. No.: US 2026/0021232 A1
(43) Pub. Date: Jan. 22, 2026

(54) SYRINGE-FREE BLOOD RETURN

(71) Applicants: Fresenius Medical Care Deutschland Gmbh, Bad Homburg (DE); Fresenius Medical Care Holdings, Inc., Waltham, MA (US)

(72) Inventors: Christian Schlaeper, Wehrheim (DE); Martin Crnkovich, Walnut Creek, CA (US); David Yuds, Hudson, NH (US)

(73) Assignees: Fresenius Medical Care Deutschland Gmbh, Bad Homburg (DE); Fresenius Medical Care Holdings, Inc., Waltham, MA (US)

(21) Appl. No.: 18/778,184

(22) Filed: Jul. 19, 2024

(51) Int. Cl. A61M 1/30; A61M 1/36; A61M 60/113

(52) U.S. Cl. CPC A61M 1/301; A61M 1/3643; A61M 1/367; A61M 60/113

(57) ABSTRACT

The present subject matter relates to systems and methods for syringe-free blood return in a dialysis machine blood circuit. A three-way connector can include a first inlet configured to be connected to a patient arterial access line, a second inlet connected to a substitution fluid line, and an outlet connected to a machine arterial line. In this arrangement, the three-way connector is configured to selectively allow a flow of substitution fluid from the substitution line into the machine arterial line and/or into the patient arterial access line without changing the connections of tubing within the circuit.

### TECHNICAL FIELD

The subject matter disclosed herein relates generally to dialysis systems and methods. More particularly, the subject matter disclosed herein relates to systems and methods for clearing blood from an arterial bloodline of a hemodialysis machine.

### BACKGROUND

Dialysis machines are known for use in the treatment of renal disease. The two principal dialysis methods are hemodialysis (HD) and peritoneal dialysis (PD). As will be readily appreciated by one of ordinary skill in the art, a hemodialysis system is arranged and configured to, inter alia, pump a patient's blood through a dialyzer. In addition, the hemodialysis system includes a HD machine and one or more containers containing a fluid (e.g., a dialysate), which during use is also pumped through the dialyzer of the HD system simultaneously with the patient's blood. A semi-permeable membrane in the dialyzer separates the blood from the dialysate within the dialyzer and allows diffusion and osmosis exchanges to take place between the dialysate and the blood. Thus arranged, in use, the patient's blood is cleaned or filtered.

At the end of a hemodialysis session, the machine's arterial bloodline is disconnected from the patient's arterial access to return the blood in the machine's arterial bloodline. A source of substitution fluid is then connected to the machine arterial line and the substitution fluid is used to rinse back the remaining blood in the tubing to the patient via the venous bloodline.

In some situations, the blood in the patient's arterial access line is either discarded or manually reinfused back into the patient using a saline-and/or substitution-fluid-filled syringe or bag. Even small amounts of blood discarded from the arterial access line may accumulate to a meaningful amount over time, however, which can require the supplementation of additional erythropoietin. Therefore, to avoid discarding any blood, clinicians perform a sequence of steps, which can include (1) clamping both the patient and machine arterial bloodlines; (2) disconnecting the arterial access line from the machine arterial line; (3) connecting a recirculating adapter to the machine arterial line; (4) connecting a substitution fluid line to the machine arterial bloodline; (5) opening the clamp on the machine arterial line; (6) running an automatic blood rinse back through the venous access; (7) filling a new, sterile syringe with saline; (8) connecting the syringe to the patient arterial access line; (9) opening the clamp on the patient arterial access line; and (10) carefully reinfusing the remaining blood back to the patient using the syringe (because there is often no air bubble detector to prevent air from being infused).

In addition to a long, cumbersome, and complex blood return procedure, manually filling the syringe with saline is time-consuming and the syringe must be discarded after each procedure. Alternatively, advanced hemodialysis machines can run the blood pump backwards at the end of the treatment to automatically return arterial blood through the patient's arterial access, and saline bag systems can take advantage of manual force to move the blood back. These solutions require additional labor, materials, and/or time, however.

It is with respect to these and other considerations that the present disclosure may be useful.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In accordance with this disclosure, systems and methods for syringe-free blood return in a dialysis machine blood circuit are provided. In one feature, a syringe-free blood return system is provided. The system can include a three-way connector having a first inlet, a second inlet, and an outlet, where the first inlet is configured to be connected to a patient arterial access line. A machine arterial line can include a first end and a second end substantially opposing the first end, where the first end is connected to the outlet of the three-way connector. A substitution fluid line can include a first end and a second end substantially opposing the first end, where the first end is connected to a substitution fluid source, and where the second end is connected to the second inlet of the three-way connector. In this arrangement, the three-way connector can be configured to selectively allow a flow of the substitution fluid into the machine arterial line and/or into the patient arterial access line.

In any preceding or subsequent embodiment, an arterial blood pump can be provided in communication with the machine arterial line and configured to pump fluid from the first end to the second end.

In any preceding or subsequent embodiment, a substitution fluid pump can be provided in communication with the substitution fluid line and configured to pump fluid from the substitution fluid source to the second inlet of the three-way connector.

In another feature, a method for syringe-free blood return in a dialysis machine blood circuit is provided that uses a three-way connector comprising a first inlet connected to a patient arterial access line, a second inlet connected to a substitution fluid line, and an outlet connected to a machine arterial line. The method can include closing the connection between the patient arterial access line and the first inlet of the three-way connector, operating both of an arterial blood pump in communication with the machine arterial line and a substitution fluid pump in communication with the substitution fluid line to rinse blood through the machine arterial line, opening the connection between the patient arterial access line and the first inlet of the three-way connector, and operating only the substitution fluid pump to rinse blood through the patient arterial access line.

In any preceding or subsequent embodiment, the three-way connector comprises a flow guide structure configured to direct fluid from each of the first inlet and the second inlet to the outlet.

In any preceding or subsequent embodiment, a cap can be selectively connectable to the first inlet, the cap being configured to prevent through the first inlet during priming of the machine arterial line.

Although some of the features of the subject matter disclosed herein have been stated hereinabove, and which are achieved in whole or in part by the presently disclosed subject matter, other features will become evident as the description proceeds when taken in connection with the accompanying drawings as best described hereinbelow.

### BRIEF DESCRIPTION OF THE DRAWINGS

The features and advantages of the present subject matter will be more readily understood from the following detailed description which should be read in conjunction with the accompanying drawings that are given merely by way of explanatory and non-limiting embodiment, and in which:

FIG. 1 is a plan view of a dialysis machine blood circuit including a syringe-free blood return system according to an embodiment of the presently disclosed subject matter.

FIGS. 2 and 3 are side cutaway views of a three-way connector configured for use in various configurations of a syringe-free blood return system according to embodiments of the presently disclosed subject matter.

FIG. 4 is a flow chart illustrating a method for syringe-free blood return in a dialysis machine blood circuit according to an embodiment of the presently disclosed subject matter.

It should be understood that the drawings are not necessarily to scale and that the disclosed embodiments are sometimes illustrated diagrammatically and in partial views. In certain instances, details which are not necessary for an understanding of the disclosed methods and devices or which render other details difficult to perceive may have been omitted. It should be further understood that this disclosure is not limited to the particular embodiments illustrated herein. In the drawings, like numbers refer to like elements throughout unless otherwise noted.

### DETAILED DESCRIPTION

Embodiments of the present disclosure will now be described more fully hereinafter with reference to the accompanying drawings, in which several exemplary embodiments are shown. The subject matter of the present disclosure, however, may be embodied in many different forms and types of methods, systems, and devices for dialysis treatments and other potential medical devices and treatments, and should not be construed as limited to the embodiments set forth herein. Rather, these embodiments are provided so that this disclosure will be thorough and complete, and willfully convey the scope of the subject matter to those skilled in the art.

The present subject matter provides syringe-free blood return systems and methods for clearing blood from an arterial bloodline of a hemodialysis machine. Various features or the like of a syringe-free blood return will now be described more fully herein with reference to the accompanying drawings, in which one or more features of the syringe-free blood return will be shown and described. It should be appreciated that the various features may be used independently of, or in combination, with each other. It will be appreciated that the syringe-free blood return as disclosed herein may be embodied in many different forms and may selectively include one or more concepts, features, or functions described herein. As such, the syringe-free blood return should not be construed as being limited to the specific embodiments set forth herein. Rather, these embodiments are provided so that this disclosure will convey certain features of the syringe-free blood return to those skilled in the art.

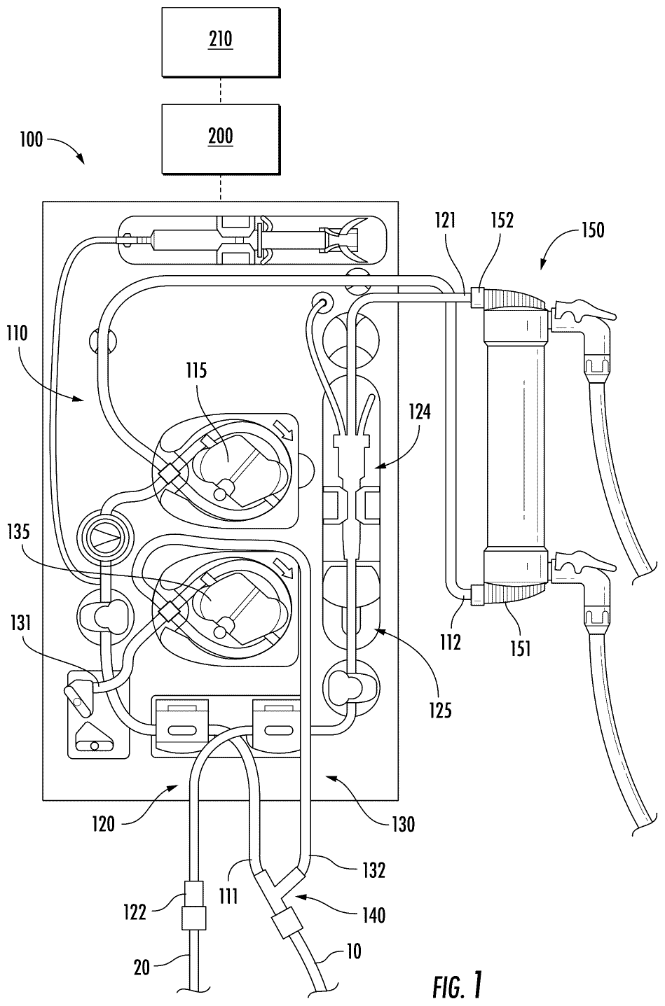

In one feature, the present subject matter provides a tubing system that connects a substitution fluid line to a three-way connection between the patient end of the arterial bloodline and the machine arterial bloodline. Further Y- or T-connections or other ports that are conventionally included upstream from the patient end can thus be eliminated for cost savings. Referring to an embodiment show in FIG. 1, the syringe-free blood return can be integrated into a dialysis machine blood circuit, generally designated 100, which can include a machine arterial line 110 having a first end 111 that is configured to be connected to a patient arterial access line 10 and a second end 112 substantially opposing the first end 111.

In some embodiments, an arterial blood pump 115 connected to the machine arterial line 110 is configured to pump the blood through the machine arterial line 110. The machine arterial line 110 transports blood from its connection to the patient arterial access line 10 at the first end 111 to the second end 112, which is configured to be connected to an input port 151 of a dialyzer, generally designated 150. Blood supplied to the dialyzer 150 can thereby be cleaned or filtered through diffusion and osmosis exchanges between the blood and a dialysate that is simultaneously pumped through the dialyzer 150. Operation of one or more components of the machine blood circuit 100 can be controlled by a control system 200, and a user interface 210 can be connected to the control system 200. In this way, operation of the machine blood circuit 100 can be easily managed by the operator and/or the operator can be prompted to perform various steps in the operation of the machine blood circuit 100.

After processing by the dialyzer 150, the blood can then be transported from an output port 152 of the dialyzer 150 to a first end 121 of a machine venous line 120 connected thereto. A second end 122 of the machine venous line 120 substantially opposing the first end 121 can then further be connected to a patient venous access line 20 to return the processed blood to the patient. In some embodiments, the machine venous line 120 includes a venous drip chamber 124 and/or an air bubble detector 125 that is configured to regulate blood flow and detect entrained air within the blood flow at a designed position between the first end 121 and the second end 122 of the machine venous line 120.

At the end of a hemodialysis session, the machine blood circuit 100 can be configured to clear blood from the machine arterial line 110 and the machine venous line 120 without reconfiguring any tubing connections or connecting a new, sterile syringe to the circuit. Instead, a substitution fluid line 130 can be connected by a three-way connector 140 to the machine arterial line 110 at or near the first end 111. In some embodiments, the substitution fluid line 130 includes a first end 131 that is connected to a source of substitution fluid and a second end 132 substantially opposing the first end 131 that is connected to the three-way connector 140. In some embodiments, the source of substitution fluid is generated onboard the machine. In some embodiments, a substitution fluid pump 135 connected to the substitution fluid line 130 is configured to pump the substitution fluid through the substitution fluid line 130. Alternatively, in some embodiments, the substitution fluid can be delivered from a saline bag or other external fluid source.

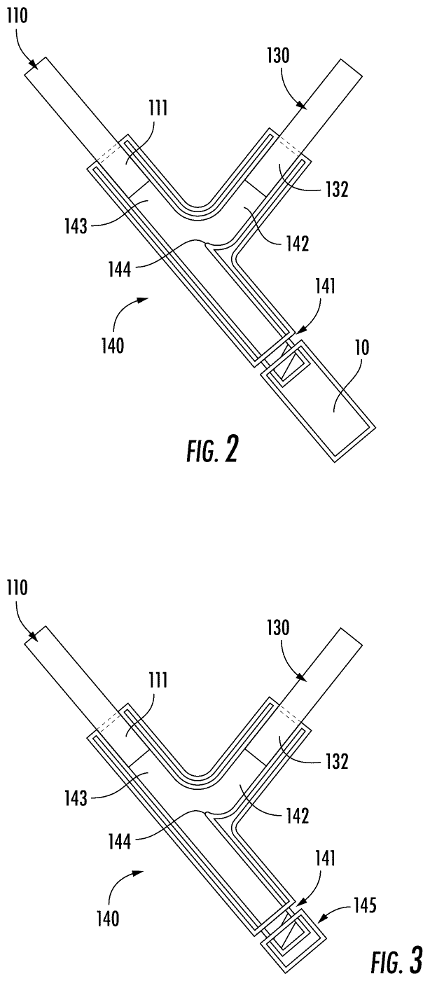

As shown in FIG. 2, the three-way connector 140 can include a first inlet 141 that is configured to be connected to the patient arterial access line 10, such as by using a Luer-lock-style connection, a second inlet 142 that is configured to be connected to the second end 132 of the substitution fluid line 130, and an outlet 143 that is in communication with both of the first inlet 141 and the second inlet 142 and is connected to the first end 111 of the machine arterial line 110. In some embodiments, the three-way connector 140 includes one or more features, such as a flow guide 144, that directs fluid flows from each of the first inlet 141 and the second inlet 142 towards the outlet 143. In this configuration, the substantially Y-shaped design of the three-way connector 140 helps to ensure that one or both of the arterial blood pump 115 or the substitution fluid pump 135 can be operated to control the relative flow of blood and/or substitution fluid to the machine arterial line 110.

Alternatively, in some embodiments, the three-way connector 140 can be provided in any of a variety of other connecter configurations that allow communication among multiple ports, such as a T-connector (e.g., a dead-space free T-connector). In some embodiments, however, it is believed that the shape of the three-way connector 140 as having a substantially Y-shaped configuration provides particular advantages for this application of controlling flows through a hemodialysis system. For example, the flow paths provided by the three-way connector 140 including the flow guide 144 can be considered more “gentle” on red blood cells passed through the three-way connector 140, and thus the risk of hemolysis can be reduced. In addition, the non-symmetric shape can help the user identify the respective flow paths more readily.

For example, when blood flows from the patient arterial access line 10 through the first inlet 141, it can travel along a straight path to the outlet 143 and the machine arterial line 110 while substitution fluid in the substitution fluid line 130 remains in place until the substitution fluid pump 135 is operated. In some embodiments, the positive fluid pressure in the substitution fluid line 130 from the stopped substitution fluid pump during treatment can be sufficient to prevent the blood from flowing into the second inlet 142 and the substitution fluid line 130. Then, during operation of both of the arterial blood pump 115 and the substitution fluid pump 135, the curve of the flow guide 144 in the three-way connector 140 ensures that substitution fluid moves toward the outlet 143 and the machine arterial line 110. When the arterial blood pump 115 is stopped, however, the running substitution fluid pump 135 is able to overcome the torturous path and be directed to the first inlet 141 and the patient arterial access line 10.

In some embodiments, the second inlet 142 of the three-way connector 140 can be bonded to the substitution fluid line and/or the outlet 143 can be bonded to the machine arterial line 110 because there is no longer any need to disconnect the lines for different phases of operation of the dialysis machine blood circuit 100. In some embodiments, for example, the ports of the three-way connector 140 can each be bonded to the connected lines using adhesives, ultrasonic bonding, or any of a variety of other fixation to secure the lines to the respective ports of the three-way connector 140. In some embodiments, however, if post-dilution of the blood at the venous drip chamber 124 is still desirable as an option, the substitution fluid line 130 can be selectively dis-connectable from the second inlet 142 of the three-way connector 140, such as by using a further Luer-lock-style connection.

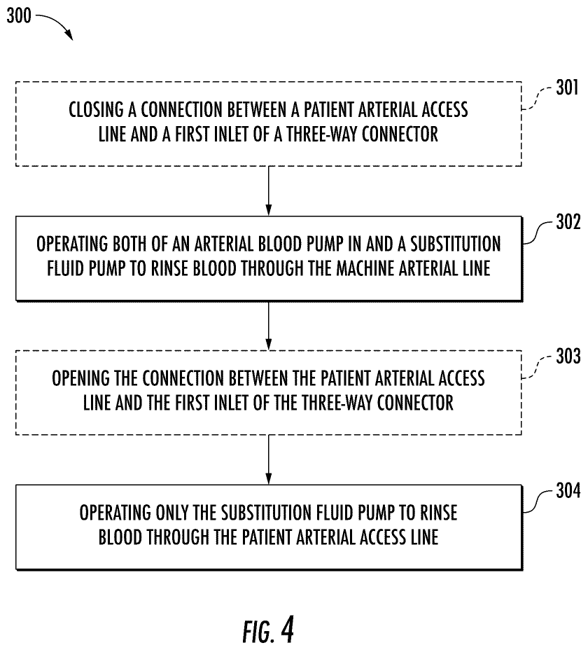

Regardless of the particular configuration, the present dialysis machine blood circuit 100 is operable such that substitution fluid can be used to rinse any remaining blood in the tubing back to the patient via the venous bloodline using a process that is dramatically simplified compared to conventional methods. Referring to FIG. 4, such a process, generally designated 300, can include an isolation step 301 of clamping the patient arterial access line 10 directly below the three-way connector 140 (i.e., at the first inlet). A machine rinse step 302 can involve running both of the arterial blood pump 115 and the substitution fluid pump 135 together to rinse blood back through the entire dialysis machine blood circuit 100 (i.e., through each of the machine arterial line 110, the dialyzer 150, the machine venous line 120, and the patient venous access line 20). A reopening step 303 can include opening the clamp again on the patient arterial access line 10. Then, an access line rinse step 304 can involve running only the substitution fluid pump 135 to rinse blood back through the patient arterial access line 10. In some embodiments, an arterial occlusion clamp 114 can be engaged prior to the access line rinse step 304 such that, while only the substitution fluid pump 135 runs, the fluid is prevented from moving through the outlet 143 of the three-way connector 140 while blood is rinsed back through the patient arterial access line 10.

Alternatively, in some embodiments, the isolation step 301 and the reopening step 303 can be omitted such that the patient arterial access line 10 can be rinsed without closing/opening the connection to the patient arterial access line 10. In such a configuration, the machine rinse step 302 can involve running the arterial blood pump 115 and the substitution fluid pump 135 at substantially the same speed such that no blood is drawn from the patient. Then, the access line rinse step 304 can involve stopping the operation of the arterial blood pump 115 while continuing to operate the substitution fluid pump 135 to flow the substitution fluid into the patient. In this way, in some embodiments, the blood return could run without a nurse attending the procedure.

With the configuration of the dialysis machine blood circuit 100 discussed herein, because the lines can remain connected during each operating phase of the machine, the possibility of introducing air is into the system is reduced. As a result, in some embodiments, the substitution fluid pump 135 can be configured to run for a pre-determined amount of time based on a known volume of fluid in the machine arterial line 110 and/or the patient arterial access line 10. The operator may trigger the machine beyond this pre-determined amount of time, however, to infuse more substitution fluid as needed. In some embodiments, the hemodialysis machine can be configured to prompt the operator when to clamp the lines and when each phase of the blood return process is complete. Upon completion of the blood return process, both of the patient arterial access line 10 and the patient venous access line 20 can be disconnected at the same time. In this way, by utilizing the three-way connector 140, fewer steps are required to return the blood to the patient. As a result, less labor, materials, and time is used while improving safety, making the blood return process almost totally automatic.

In a further feature, the configuration of the dialysis machine blood circuit 100 including the three-way connector 140 can be used to prime the dialysis machine blood circuit 100 before use. Referring to FIG. 3, in some embodiments, the first inlet 141 of the three-way connector 140 can be blocked prior to connecting the first inlet 141 to the patient arterial access line 10, such as by attaching a cap 145 to the first inlet 141. In this configuration, the substitution fluid pump 135 can be operated to prime the dialysis machine blood circuit 100 with the substitution fluid. Once the system is primed, the cap 145 can be removed, and the first inlet 141 can be connected to the patient arterial access line 10.

The present subject matter can be embodied in other forms without departure from the spirit and essential characteristics thereof. The embodiments described therefore are to be considered in all respects as illustrative and not restrictive. Although the present subject matter has been described in terms of certain preferred embodiments, other embodiments that are apparent to those of ordinary skill in the art are also within the scope of the present subject matter.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A syringe-free blood return system comprising:
a three-way connector comprising a first inlet, a second inlet, and an outlet, wherein the first inlet is configured to be connected to a patient arterial access line;
a machine arterial line comprising a first end and a second end substantially opposing the first end, wherein the first end is connected to the outlet of the three-way connector; and
a substitution fluid line comprising a first end and a second end substantially opposing the first end, wherein the first end is connected to a substitution fluid source, and wherein the second end is connected to the second inlet of the three-way connector;
wherein the three-way connector is configured to selectively allow a flow of the substitution fluid into the machine arterial line or into the patient arterial access line.

2. The system of claim 1, wherein the three-way connector comprises a flow guide structure configured to direct fluid from each of the first inlet and the second inlet to the outlet.

3. The system of claim 1, comprising an arterial blood pump in communication with the machine arterial line and configured to pump fluid from the first end to the second end.

4. The system of claim 1, comprising a substitution fluid pump in communication with the substitution fluid line and configured to pump fluid from the substitution fluid source to the second inlet of the three-way connector.

5. The system of claim 1, comprising a cap that is selectively connectable to the first inlet and that is configured to prevent through the first inlet during priming of the machine arterial line.

6. A dialysis machine comprising:
a three-way connector comprising a first inlet, a second inlet, and an outlet, wherein the first inlet is configured to be connected to a patient arterial access line;
a machine arterial line comprising a first end and a second end substantially opposing the first end, wherein the first end is connected to the outlet of the three-way connector;
an arterial blood pump in communication with the machine arterial line and configured to pump fluid from the first end to the second end;
a substitution fluid line comprising a first end and a second end substantially opposing the first end, wherein the first end is connected to a substitution fluid source, and wherein the second end is connected to the second inlet of the three-way connector; and
a substitution fluid pump in communication with the substitution fluid line and configured to pump fluid from the substitution fluid source to the second inlet of the three-way connector;
wherein the arterial blood pump and the substitution fluid pump are selectively operable to provide a flow of the substitution fluid into the machine arterial line or into the patient arterial access line.

7. A method for syringe-free blood return in a dialysis machine blood circuit using a three-way connector comprising a first inlet connected to a patient arterial access line, a second inlet connected to a substitution fluid line, and an outlet connected to a machine arterial line, the method comprising:
operating both of an arterial blood pump in communication with the machine arterial line and a substitution fluid pump in communication with the substitution fluid line to rinse blood through the machine arterial line; and
operating only the substitution fluid pump to rinse blood through the patient arterial access line.

8. The method of claim 7, wherein operating both of the arterial blood pump and the substitution fluid pump comprises operating the substitution fluid pump for a predetermined amount of time based on a known volume of fluid in the machine arterial line.

9. The method of claim 7, wherein operating only the substitution fluid pump comprises engaging an arterial occlusion clamp in communication with the machine arterial line to prevent fluid from moving through the outlet of the three-way connector.

10. The method of claim 7, wherein operating only the substitution fluid pump comprises operating the substitution fluid pump for a predetermined amount of time based on a known volume of fluid in the patient arterial access line.

11. The method of claim 7, comprising closing the connection between the patient arterial access line and the first inlet of the three-way connector prior to operating both of the arterial blood pump and the substitution fluid pump to rinse blood through the machine arterial line.

12. The method of claim 11, comprising opening the connection between the patient arterial access line and the first inlet of the three-way connector after operating both of the arterial blood pump and the substitution fluid pump to rinse blood through the machine arterial line.
